(19) United States
(12) Patent Application Publication
(10) Pub. No.: US 2026/0174331 A1
(43) Pub. Date: Jun. 25, 2026

(54) INDICATOR EVALUATION DEVICE FOR HUMAN 5.0T MULTI-NUCLEAR MAGNETIC RESONANCE IMAGING

(71) Applicant: INNOVATION ACADEMY FOR PRECISION MEASUREMENT SCIENCE AND TECHNOLOGY, CAS, Wuhan (CN)

(72) Inventors: Xin ZHOU, Wuhan (CN); Yingying LUO, Wuhan (CN); Sa XIAO, Wuhan (CN)

(73) Assignee: INNOVATION ACADEMY FOR PRECISION MEASUREMENT SCIENCE AND TECHNOLOGY, CAS, Wuhan (CN)

(21) Appl. No.: 19/422,483

(22) Filed: Dec. 17, 2025

(30) Foreign Application Priority Data
Dec. 19, 2024 (CN) 2024118761496

(51) Int. Cl. A61B 5/00

(52) U.S. Cl. CPC A61B 5/0035; A61B 5/4064; A61B 5/742

(57) ABSTRACT

The provided is an indicator evaluation device for human 5.0 T multi-nuclear MRI, including a clinical indicator module, a multi-nuclear indicator module, a main control module, a display module, and an indicator accuracy evaluation module. The indicator accuracy evaluation module constructs a brain multi-nuclear indicator evaluation module, trains the brain multi-nuclear indicator evaluation module based on a training set, and inputs a sample group to be identified into the trained brain multi-nuclear indicator evaluation module to obtain predicted scores. The indicator evaluation device employs a dual-channel transformer module that utilizes self-attention in non-overlapping local windows, reducing computational costs and enabling better focus on local information. The indicator evaluation device enables the collection of multi-nuclear indicators obtained from human 5.0 T multi-nuclear MRI while synchronously collecting clinical indicators, thereby evaluating the accuracy of 5.0 T multi-nuclear MRI indicators to ensure the reliability and stability of the human 5.0 T multi-nuclear MRI system.

### CROSS-REFERENCE TO THE RELATED APPLICATIONS

This application is based upon and claims priority to Chinese Patent Application No. 202411876149.6, filed on Dec. 19, 2024, the entire contents of which are incorporated herein by reference.

### TECHNICAL FIELD

The present invention belongs to the technical field of magnetic resonance imaging (MRI), and particularly relates to an indicator evaluation device for human 5.0 T multi-nuclear MRI.

### BACKGROUND

MRI can non-destructively acquire structural information of living organisms with high resolution, providing unparalleled capabilities for vascular, neural and diffusion functional imaging, and soft tissue resolution compared to other imaging modalities. Furthermore, MRI has become a powerful tool for clinical applications and life science research thanks to its advantages of no ionizing radiation, no tissue penetration depth limitations, and the ability to reveal kinetic information at atomic resolution.

Currently, existing human MRI equipment is exclusively single-nuclear (¹H). Besides ¹H, the human body contains other elements such as ²³Na and ³¹P that can reflect metabolic information, which have been used in research on various diseases including brain disorders. Therefore, multi-nuclear MRI represents the development direction of next-generation clinical imaging equipment and has the potential to become a new clinical non-invasive imaging method. Additionally, the lower field strength of clinical 3.0 T MRI cannot meet the sensitivity requirements for multi-nuclide detection and early disease diagnosis. Thus, higher-field 5.0 T multi-nuclear MRI equipment can further enhance sensitivity, signal-to-noise ratio, and spatial resolution, thereby expanding clinical application scenarios for multi-nuclear MRI.

Currently, a human 5.0 T MRI system has been deployed in multiple hospitals, with ongoing efforts to implement clinical human multi-nuclear MRI to capture the high ground in next-generation MRI equipment. After successful integration of the human 5.0 T multi-nuclear MRI system, comprehensive indicator evaluation methods must be established to assess the accuracy of acquired multi-nuclear indicators, ensuring the reliability and stability of the system. This will provide a basis for conducting subsequent clinical trials on 5.0 T multi-nuclear MRI and investigating correlations between 5.0 T multi-nuclear MRI indicators and diseases.

### SUMMARY

The objective of the present invention is to provide an indicator evaluation device for human 5.0 T multi-nuclear MRI, addressing the aforementioned problems in existing technologies.

The technical problem solved by the present invention may be implemented using the following technical solution:

An indicator evaluation device for human 5.0 T multi-nuclear MRI, comprising:

A clinical indicator module configured to receive clinical indicators of each volunteer;
A multi-nuclear indicator module configured to receive 5.0 T multi-nuclear MRI indicators of each brain region of each volunteer;
A main control module configured to transmit the clinical indicators acquired by the clinical indicator module and the 5.0 T multi-nuclear MRI indicators acquired by the multi-nuclear indicator module to an indicator accuracy evaluation module, and further configured to receive predicted scores from the indicator accuracy evaluation module;
An indicator accuracy evaluation module configured to construct a brain multi-nuclear indicator evaluation module and train the brain multi-nuclear indicator evaluation module based on a training set, and further configured to input a sample group to be identified into the trained brain multi-nuclear indicator evaluation module to obtain the predicted scores, wherein the training set includes a plurality of sample groups, each sample group includes clinical indicators acquired by the clinical indicator module and 5.0 T multi-nuclear MRI indicators acquired by the multi-nuclear indicator module for the same volunteer, and the 5.0 T multi-nuclear MRI indicators of each sample group correspond to a true score;
A display module configured to display the input clinical indicators and 5.0 T multi-nuclear MRI indicators in real time, and further configured to display the clinical indicators and the predicted scores corresponding to the 5.0 T multi-nuclear MRI indicators.

The brain multi-nuclear indicator evaluation module is a Transformer Model incorporating a dual-channel transformer module in a decoder.

The dual-channel transformer module described above includes a first normalization layer, a window-based multi-head self-attention module, a second normalization layer, a multi-head self-attention module, a third normalization layer, a first feed-forward neural layer, a fourth normalization layer, a self-attention module, a fifth normalization layer, and a second feed-forward neural layer;

Feature X1, output by the encoder-side down-sampling module of the Transformer Model, is sequentially processed by the first normalization layer and the window-based multi-head self-attention module, and the resulting feature is then concatenated with the original feature X1 to form the first concatenated feature,

Feature X2, output by the last multi-head self-attention sub-layer within the decoder of the Transformer Model, is sequentially processed by the second normalization layer and the multi-head attention module, and the resulting processed feature is then concatenated with the original feature X2 to form the second concatenated feature,

The first concatenated feature and the second concatenated feature are concatenated to form a third concatenated feature; the third concatenated feature is sequentially processed by the third normalization layer and the first feed-forward neural layer, and the processed feature is concatenated with the third concatenated feature to form a fourth concatenated feature; the fourth concatenated feature serves as an input feature to the fourth normalization layer; the fourth concatenated feature is sequentially processed by the fourth normalization layer and the self-attention module to form a feature that is concatenated with the fourth concatenated feature, forming a fifth concatenated feature; the fifth concatenated feature serves as an input feature to the fifth normalization layer; the fifth concatenated feature is sequentially processed by the fifth normalization layer and the second feed-forward neural layer to form a feature that is concatenated with the fifth concatenated feature, forming a sixth concatenated feature; the sixth concatenated feature is input to an output layer of the Transformer Model, and a predicted score is output through a softmax function of the output layer.

The total loss function Loss of the brain multi-nuclear indicator evaluation module described above is as follows:

$$Loss = \frac{1}{N} {\sum }_{i = 1}^{N} {{W}_{i} [ {y}_{i} - clip ( {y}_{i}^{′} , 1 , 100 ) ]}^{2}$$

Wherein N is the number of sample groups, and yᵢ^{′} is the true score of the 5.0 T multi-nuclear MRI indicators of the i-th sample group; yᵢ^{′} represents the predicted score corresponding to the i-th sample group; the clip function indicates restricting the predicted score to a range from 1 to 100; Wᵢ represents the weight of the i-th sample group.

The training of the brain multi-nuclear indicator evaluation module described above is based on minimizing the total loss function Loss.

The main control module described above is further configured to provide suggestions on whether to optimize the sampling method and sequence of the 5.0 T multi-nuclear MRI indicators according to the predicted scores and display the suggestions on the display module.

A computer device, comprising a memory and a processor, wherein the memory stores a computer program, and the processor, when executing the computer program, implements the clinical indicator module, the multi-nuclear indicator module, the main control module, the display module, and the indicator accuracy evaluation module of the aforementioned indicator evaluation device.

A computer-readable storage medium having a computer program stored thereon, wherein the computer program, when executed by a processor, implements the clinical indicator module, the multi-nuclear indicator module, the main control module, the display module, and the indicator accuracy evaluation module of the aforementioned indicator evaluation device.

A computer program product comprising a computer program that, when executed by a processor, implements the clinical indicator module, the multi-nuclear indicator module, the main control module, the display module, and the indicator accuracy evaluation module of the aforementioned indicator evaluation device.

Compared with the existing technology, the present invention exhibits the following beneficial effects:
1. The present invention improves upon the Transformer Model by designing a novel dual-channel transformer module. Unlike traditional Transformer models that employ global self-attention, this module utilizes self-attention in non-overlapping local windows, reducing computational costs while enabling better focus on local information;
2. A loss function is designed that incorporates weighting factors to better focus on scores within a specific range. Simultaneously, normalization operations are combined to constrain the output scores to a range between 1 and 100.
3. The present invention enables the collection of multi-nuclear indicators obtained from human 5.0 T multi-nuclear MRI while synchronously collecting clinical indicators, thereby evaluating the accuracy of 5.0 T multi-nuclear MRI indicators to ensure the reliability and stability of the human 5.0 T multi-nuclear MRI system. Ultimately, this will provide a basis for conducting subsequent clinical trials on 5.0 T multi-nuclear MRI and investigating correlations between 5.0 T multi-nuclear MRI indicators and diseases.

### BRIEF DESCRIPTION OF THE DRAWINGS

FIG. 1 is a schematic structural diagram of the present invention;

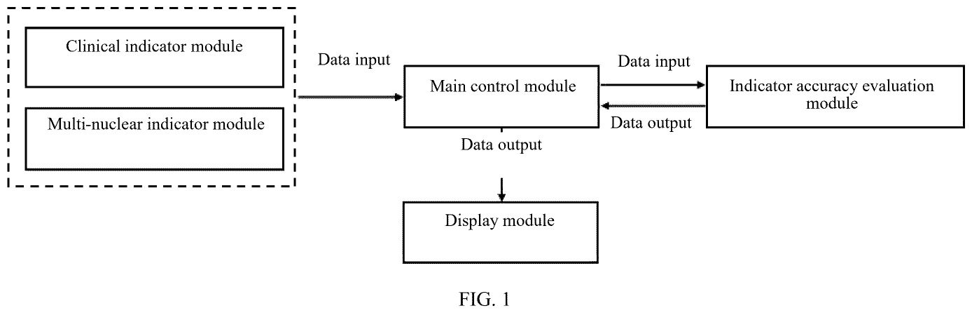

FIG. 2 is a schematic structural diagram of the dual-channel transformer module.

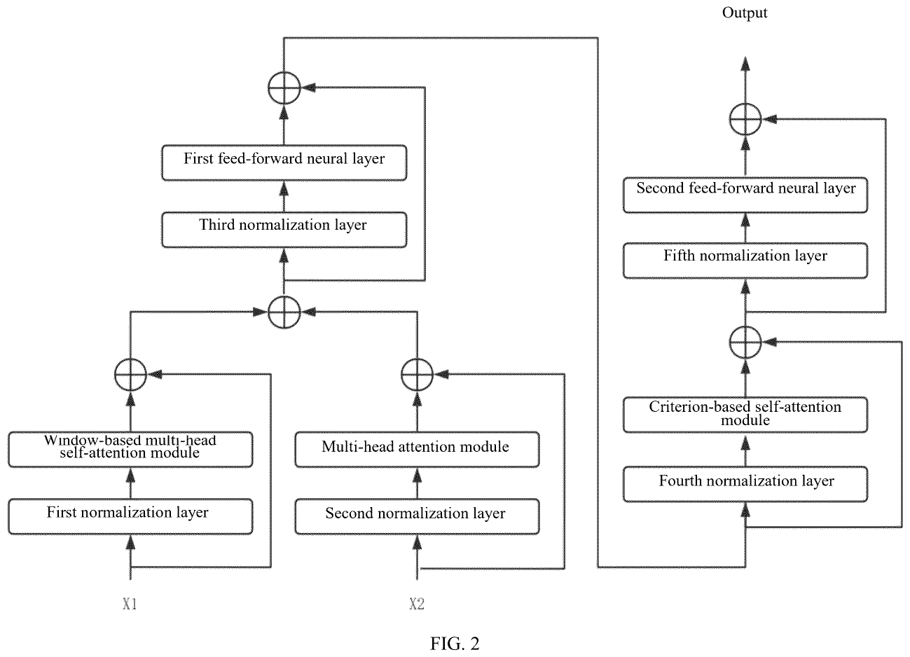

### DETAILED DESCRIPTION OF THE EMBODIMENTS

To facilitate understanding and implementation of the present invention by ordinary technicians in the field, the following provides a more detailed description of the present invention in conjunction with embodiments. It should be understood that the embodiments described herein are intended only to illustrate and explain the present invention, and not to limit it.

### Embodiment 1

An evaluation device for human brain 5.0 T multi-nuclear MRI indicators, comprising a clinical indicator module, a multi-nuclear indicator module, a main control module, a display module, and an indicator accuracy evaluation module.

The clinical indicator module is configured to receive clinical indicators for each volunteer, wherein the clinical indicators include physiological data and clinical results.

Herein, the physiological data includes gender, family history, symptoms, blood pressure, and blood oxygen levels, among others;

The clinical results include clinical diagnosis results, complete blood count (CBC), blood biochemistry, serum sodium levels, serum phosphorus levels, electroencephalogram (EEG) results, and brain disease biomarker levels, among others. The clinical results further include brain computed tomography (CT) results, low-field ¹H MRI findings, and metabolite concentration ratios in various brain regions measured by low-field ¹H magnetic resonance spectroscopy (MRS). The brain CT results include Hounsfield unit (HU) values measured by CT, among others. The low-field ¹H MRI findings include tissue homogeneity, perfusion levels, and apparent diffusion coefficient (ADC) in various brain regions. The metabolite concentration ratios in various brain regions measured by low-field ¹H MRS include choline/N-acetylaspartate (Cho/NAA) concentration ratio and N-acetylaspartate/creatine-phosphocreatine complex (NAA/Cr) concentration ratio, among others.

The multi-nuclear indicator module is configured to receive 5.0 T multi-nuclear MRI indicators for various brain regions of each volunteer, wherein the 5.0 T multi-nuclear MRI indicators include:
¹H magnetic resonance spectroscopy (MRS) indicators: such as choline/N-acetylaspartate (Cho/NAA) concentration ratio and N-acetylaspartate/creatine-phosphocreatine complex (NAA/Cr) concentration ratio;
²³Na MRI indicators: such as tissue sodium concentration (TSC) range, tissue sodium concentration (TSC) mean, and tissue sodium concentration (TSC) uniformity;
³¹P MRS indicators: ratios of various phosphorus-containing compounds, including phosphocreatine/adenosine triphosphate (PCr/ATP) ratio, inorganic phosphate/adenosine triphosphate (Pi/ATP) ratio, phosphocreatine/inorganic phosphate (PCr/Pi) ratio, and phosphomonoester/phosphodiesterase (PME/PDE) ratio;
And the N-acetylaspartate/tissue sodium concentration (NAA/TSC) ratio acquired by combining ¹H MRS and ²³Na MRI.

A main control module configured to transmit the clinical indicators acquired by the clinical indicator module and the 5.0 T multi-nuclear MRI indicators acquired by the multi-nuclear indicator module to an indicator accuracy evaluation module, and further configured to receive predicted scores from the indicator accuracy evaluation module.

A display module configured to display the input clinical indicators and 5.0 T multi-nuclear MRI indicators in real time to facilitate viewing and modification by technicians, and further configured to display the clinical indicators and the predicted scores corresponding to the 5.0 T multi-nuclear MRI indicators.

An indicator accuracy evaluation module configured to construct a brain multi-nuclear indicator evaluation module and train the brain multi-nuclear indicator evaluation module based on a training set, and further configured to input a sample group to be identified into the trained brain multi-nuclear indicator evaluation module to obtain the predicted scores. The training set includes a plurality of sample groups, each sample group includes clinical indicators acquired by the clinical indicator module and 5.0 T multi-nuclear MRI indicators acquired by the multi-nuclear indicator module for the same volunteer, and the 5.0 T multi-nuclear MRI indicators of each sample group correspond to a true score.

The brain multi-nuclear indicator evaluation module improves upon the Transformer Model. The Transformer Model includes an input layer, an encoder, a decoder, and an output layer. A dual-channel transformer module is introduced into the decoder of the Transformer Model to enhance the performance of the brain multi-nuclear indicator evaluation model in processing sequence data or image data. The dual-channel transformer module includes a first normalization layer, a window-based multi-head self-attention module, a second normalization layer, a multi-head self-attention module, a third normalization layer, a first feed-forward neural layer, a fourth normalization layer, a self-attention module, a fifth normalization layer, and a second feed-forward neural layer. The dual-channel transformer module first splits into two paths, respectively receiving feature X1 output by the encoder-side down-sampling module of the Transformer Model and feature X2 last output by the multi-head self-attention module in the decoder of the Transformer Model. One path uses conventional multi-head self-attention, while the other uses window-based multi-head self-attention. Feature X1 is processed sequentially through the first normalization layer and the window-based multi-head self-attention module, and the processed feature is concatenated with feature X1 to form a first concatenated feature; feature X2 is processed sequentially through the second normalization layer and the multi-head attention module, and the processed feature is concatenated with feature X2 to form a second concatenated feature; the first concatenated feature and the second concatenated feature are concatenated to form a third concatenated feature; the third concatenated feature is processed sequentially through the third normalization layer and the first feed-forward neural layer, and the processed feature is concatenated with the third concatenated feature to form a fourth concatenated feature; the fourth concatenated feature serves as an input to the fourth normalization layer; the fourth concatenated feature is processed sequentially through the fourth normalization layer and the self-attention module, and the processed feature is concatenated with the fourth concatenated feature to form a fifth concatenated feature; the fifth concatenated feature serves as an input to the fifth normalization layer; the fifth concatenated feature is processed sequentially through the fifth normalization layer and the second feed-forward neural layer, and the processed feature is concatenated with the fifth concatenated feature to form a sixth concatenated feature; the sixth concatenated feature serves as an output feature of the dual-channel transformer module. The output feature of the dual-channel transformer module is input to the output layer of the Transformer Model, and a predicted score is output through a softmax function of the output layer.

The total loss function Loss of the brain multi-nuclear indicator evaluation module is as shown in the formula:

$$Loss = \frac{1}{N} {\sum }_{i = 1}^{N} {{W}_{i} [ {y}_{i} - clip ( {y}_{i}^{′} , 1 , 100 ) ]}^{2}$$

Wherein N is the number of sample groups input to the brain multi-nuclear indicator evaluation module, yᵢ is the true score of the 5.0 T multi-nuclear MRI indicators for the i-th sample group (constrained to the range of 1 to 100); yᵢ^{′} represents the predicted score corresponding to the i-th sample group; the clip function indicates restricting the predicted score to a range from 1 to 100; Wᵢ represents the weight of the i-th sample group, which can be dynamically adjusted based on the position of the target score, and samples close to boundaries can be assigned greater weights.

Based on minimizing the total loss function, the brain multi-nuclear indicator evaluation module is trained end-to-end using the training set;

The network learning rate is initialized to 0.0001, the batch size is set to 8, and the Adam optimizer is used for network training on the PyTorch platform. The sample groups from the training set are input into the brain multi-nuclear indicator evaluation module to obtain prediction results, with network training conducted throughout according to the total loss function set in Step 3. Network training stops after reaching a total of 200 iterations, and the network parameters corresponding to the brain multi-nuclear indicator evaluation module are saved.

The sample group to be identified is input into the trained brain multi-nuclear indicator evaluation module to obtain its predicted score, which is then output to the display module via the main control module for display.

The main control module is further configured to provide suggestions on whether to optimize the sampling method and sequence for the 5.0 T multi-nuclear MRI indicators according to the predicted score and display the suggestions on the display module. If the predicted score obtained from the sample group to be identified is no more than 60 (inclusive), the technician will be prompted to optimize the sampling method and sequence for the 5.0 T multi-nuclear MRI indicators of the sample group to be identified; if the predicted score is more than 60, the technician will be prompted that the 5.0 T multi-nuclear MRI indicators of the sample group to be identified features certain accuracy and can proceed to the next step of 5.0 T multi-nuclear MRI clinical trials to investigate the correlation between these indicators and brain diseases.

Ordinary technicians in the field will understand that all or part of the processes for implementing the clinical indicator module, the multi-nuclear indicator module, the main control module, the display module, and the indicator accuracy evaluation module in the above embodiments may be accomplished by a computer program instructing relevant hardware. The computer program may be stored in a non-volatile computer-readable storage medium. When executed, the computer program may include the processes of the aforementioned clinical indicator module, multi-nuclear indicator module, main control module, display module, and indicator accuracy evaluation module.

### Embodiment 2

In this embodiment, a computer device comprising a memory and a processor is also provided, wherein the memory stores a computer program and the processor, when executing the computer program, implements the clinical indicator module, the multi-nuclear indicator module, the main control module, the display module, and the indicator accuracy evaluation module of Embodiment 1 above.

### Embodiment 3

In this embodiment, a computer-readable storage medium is provided, having a computer program stored thereon that, when executed by a processor, implements the clinical indicator module, multi-nuclear indicator module, the main control module, the display module, and the indicator accuracy evaluation module of Embodiment 1 above.

### Embodiment 4

In this embodiment, a computer program product is provided, comprising a computer program that, when executed by a processor, implements the clinical indicator module, the multi-nuclear indicator module, the main control module, the display module, and the indicator accuracy evaluation module of Embodiment 1 above.

It should be noted that the embodiments described in the present invention are merely illustrative of the spirit of the invention. Technicians in the field to which the present invention pertains may make various modifications or supplements of the described embodiments or substitute them using similar methods, but such actions shall not depart from the spirit of the invention or exceed the scope defined by the appended claims.

What is claimed is:

1. An indicator evaluation device for human 5.0 T multi-nuclear magnetic resonance imaging (MRI), comprising:
a clinical indicator module configured to receive clinical indicators of each volunteer;
a multi-nuclear indicator module configured to receive 5.0 T multi-nuclear MRI indicators of each brain region of each volunteer;
a main control module configured to transmit the clinical indicators acquired by the clinical indicator module and the 5.0 T multi-nuclear MRI indicators acquired by the multi-nuclear indicator module to an indicator accuracy evaluation module, and further configured to receive predicted scores from the indicator accuracy evaluation module;
an indicator accuracy evaluation module configured to construct a brain multi-nuclear indicator evaluation module and train the brain multi-nuclear indicator evaluation module based on a training set, and further configured to input a sample group to be identified into a trained brain multi-nuclear indicator evaluation module to obtain predicted scores, wherein the training set comprises a plurality of sample groups, each sample group comprises clinical indicators acquired by the clinical indicator module and 5.0 T multi-nuclear MRI indicators acquired by the multi-nuclear indicator module for the same volunteer, and the 5.0 T multi-nuclear MRI indicators of each sample group correspond to a true score;
a display module configured to display input clinical indicators and 5.0 T multi-nuclear MRI indicators in real time, and further configured to display the clinical indicators and the predicted scores corresponding to the 5.0 T multi-nuclear MRI indicators;
wherein the brain multi-nuclear indicator evaluation module is a transformer model incorporating a dual-channel transformer module in a decoder;
wherein the dual-channel transformer module comprises a first normalization layer, a window-based multi-head self-attention module, a second normalization layer, a multi-head self-attention module, a third normalization layer, a first feed-forward neural layer, a fourth normalization layer, a self-attention module, a fifth normalization layer, and a second feed-forward neural layer;
feature X1, output by an encoder-side down-sampling module of the Transformer Model, is sequentially processed by the first normalization layer and the window-based multi-head self-attention module, and a resulting feature is then concatenated with an original feature X1 to form a first concatenated feature;
feature X2, finally output by a multi-head self-attention module within the decoder of the Transformer Model, is sequentially processed by the second normalization layer and the multi-head attention module, and a resulting feature is then concatenated with an original feature X2 to form a second concatenated feature;
the first concatenated feature and the second concatenated feature are concatenated to form a third concatenated feature; the third concatenated feature is sequentially processed by the third normalization layer and the first feed-forward neural layer, and a processed feature is concatenated with the third concatenated feature to form a fourth concatenated feature; the fourth concatenated feature serves as an input feature to the fourth normalization layer; the fourth concatenated feature is sequentially processed by the fourth normalization layer and the self-attention module to form a feature that is concatenated with the fourth concatenated feature, forming a fifth concatenated feature; the fifth concatenated feature serves as an input feature to the fifth normalization layer; the fifth concatenated feature is sequentially processed by the fifth normalization layer and the second feed-forward neural layer to form a feature that is concatenated with the fifth concatenated feature, forming a sixth concatenated feature; the sixth concatenated feature is input to an output layer of the Transformer Model, and a predicted score is output through a softmax function of the output layer.

2. The indicator evaluation device for the human 5.0 T multi-nuclear MRI according to claim 1, wherein a total loss function Loss of the brain multi-nuclear indicator evaluation module is:
$$Loss = \frac{1}{N} {\sum }_{i = 1}^{N} {{W}_{i} [ {y}_{i} - clip ( {y}_{i}^{′} , 1 , 100 ) ]}^{2} ;$$
wherein N is a number of sample groups, and yᵢ is the true score of the 5.0 T multi-nuclear MRI indicators of an i-th sample group; yᵢ^{′} represents the predicted score corresponding to the i-th sample group; the clip function indicates restricting the predicted score to a range from 1 to 100; Wᵢ represents a weight of the i-th sample group.

3. The indicator evaluation device for the human 5.0 T multi-nuclear MRI according to claim 2, wherein training of the brain multi-nuclear indicator evaluation module is based on minimizing the total loss function Loss.

4. The indicator evaluation device for the human 5.0 T multi-nuclear MRI according to claim 1, wherein the main control module is further configured to provide suggestions on whether to optimize a sampling method and sequence of the 5.0 T multi-nuclear MRI indicators according to the predicted scores and display the suggestions on the display module.

5. A computer device comprising a memory and a processor, wherein the memory stores a computer program, and the processor, when executing the computer program, implements the clinical indicator module, the multi-nuclear indicator module, the main control module, the display module, and the indicator accuracy evaluation module of the indicator evaluation device according to claim 1.

6. A computer-readable storage medium having a computer program stored thereon, wherein when the computer program is executed by a processor, it implements the clinical indicator module, the multi-nuclear indicator module, the main control module, the display module, and the indicator accuracy evaluation module of the indicator evaluation device according to claim 1.

7. A computer program product comprising a computer program, wherein when the computer program is executed by a processor, it implements the clinical indicator module, the multi-nuclear indicator module, the main control module, the display module, and the indicator accuracy evaluation module of the indicator evaluation device according to claim 1.

8. The computer device according to claim 5, wherein in the indicator evaluation device, a total loss function Loss of the brain multi-nuclear indicator evaluation module is:
$$Loss = \frac{1}{N} {\sum }_{i = 1}^{N} {{W}_{i} [ {y}_{i} - clip ( {y}_{i}^{′} , 1 , 100 ) ]}^{2} ;$$
wherein N is a number of sample groups, and yᵢ is the true score of the 5.0 T multi-nuclear MRI indicators of an i-th sample group; yᵢ^{′} represents the predicted score corresponding to the i-th sample group; the clip function indicates restricting the predicted score to a range from 1 to 100; Wᵢ represents a weight of the i-th sample group.

9. The computer device according to claim 8, wherein in the indicator evaluation device, training of the brain multi-nuclear indicator evaluation module is based on minimizing the total loss function Loss.

10. The computer device according to claim 5, wherein in the indicator evaluation device, the main control module is further configured to provide suggestions on whether to optimize a sampling method and sequence of the 5.0 T multi-nuclear MRI indicators according to the predicted scores and display the suggestions on the display module.

11. The computer-readable storage medium according to claim 6, wherein in the indicator evaluation device, a total loss function Loss of the brain multi-nuclear indicator evaluation module is:
$$Loss = \frac{1}{N} {\sum }_{i = 1}^{N} {{W}_{i} [ {y}_{i} - clip ( {y}_{i}^{′} , 1 , 100 ) ]}^{2} ;$$
wherein N is a number of sample groups, and yᵢ is the true score of the 5.0 T multi-nuclear MRI indicators of an i-th sample group; yᵢ^{′} represents the predicted score corresponding to the i-th sample group; the clip function indicates restricting the predicted score to a range from 1 to 100; Wᵢ represents a weight of the i-th sample group.

12. The computer-readable storage medium according to claim 11, wherein in the indicator evaluation device, training of the brain multi-nuclear indicator evaluation module is based on minimizing the total loss function Loss.

13. The computer-readable storage medium according to claim 6, wherein in the indicator evaluation device, the main control module is further configured to provide suggestions on whether to optimize a sampling method and sequence of the 5.0 T multi-nuclear MRI indicators according to the predicted scores and display the suggestions on the display module.

14. The computer program product according to claim 7, wherein in the indicator evaluation device, a total loss function Loss of the brain multi-nuclear indicator evaluation module is:
$$Loss = \frac{1}{N} {\sum }_{i = 1}^{N} {{W}_{i} [ {y}_{i} - clip ( {y}_{i}^{′} , 1 , 100 ) ]}^{2} ;$$
wherein N is a number of sample groups, and yᵢ is the true score of the 5.0 T multi-nuclear MRI indicators of an i-th sample group; yᵢ^{′} represents the predicted score corresponding to the i-th sample group; the clip function indicates restricting the predicted score to a range from 1 to 100; Wᵢ represents a weight of the i-th sample group.

15. The computer program product according to claim 14, wherein in the indicator evaluation device, training of the brain multi-nuclear indicator evaluation module is based on minimizing the total loss function Loss.

16. The computer program product according to claim 7, wherein in the indicator evaluation device, the main control module is further configured to provide suggestions on whether to optimize a sampling method and sequence of the 5.0 T multi-nuclear MRI indicators according to the predicted scores and display the suggestions on the display module.
